(19) United States
(12) Patent Application Publication
(10) Pub. No.: US 2024/0122175 A1
(43) Pub. Date: Apr. 18, 2024

(54) ENHANCED FRAGRANCE-EMITTING COMPOSITION AND ARTICLES UTILIZING THE SAME

(71) Applicant: Clyde Morgan, Gardner, KS (US)

(72) Inventor: Clyde Morgan, Gardner, KS (US)

(21) Appl. No.: 18/484,784

(22) Filed: Oct. 11, 2023

Related U.S. Application Data
(60) Provisional application No. 63/415,851, filed on Oct. 13, 2022.

(51) Int. Cl. A01N 25/08; A01N 65/06; A01N 65/08; A01N 65/22; A01N 65/24; A01N 65/28; A01N 65/42; A01P 17/00; A61L 9/04; A61L 9/12

(52) U.S. Cl. CPC A01N 25/08; A01N 65/06; A01N 65/08; A01N 65/22; A01N 65/24; A01N 65/28; A01N 65/42; A01P 17/00; A61L 9/046; A61L 9/12

(57) ABSTRACT

Fragrant compositions including a blend of essential oils suitable for use in fragrance-emitting articles are provided herein. Articles formed by infusing such compositions into porous substrates, such as rock, are also provided according to aspects of the present technology, and these may be particularly useful as an animal repellant in both indoor and outdoor spaces.

### CROSS-REFERENCE TO RELATED APPLICATION

The present application claims priority to U.S. Provisional Application Ser. No. 63/415,851, filed on Oct. 13, 2022, the entire disclosure of which is incorporated herein by reference.

### FIELD OF THE INVENTION

The present technology generally relates methods of making and using fragrant compositions. More specifically, embodiments of the present technology concern blends of essential oils and articles utilizing the same that can be used, for example, to control the activity of one or more species of animal.

### BACKGROUND

Products and devices for emitting fragrance into an environment are used in a wide variety of applications. For example, air fresheners, diffusers, misters, and the like are used in many indoor environments to impart certain fragrances to a space. Outdoors, scents like citronella can be used to repel insects including mosquitos. However, conventional scent-emitting devices and methods have drawbacks. For example, some fragrances are formed with synthetic chemicals, which can pose a health risk to animals, plants, and/or humans. Other fragrance emitting devices are electric and require a power source to operate. Additionally, loose particles associated with some scent-emitting compositions pose a risk for eye irritation, or the shape and/or smell of some compositions or articles may pose a risk for accidental consumption by animals, birds, or even small children. Other scent-emitting devices may not be robust enough for use in more severe environments, such as outdoors, where wind, rain, and even UV light may degrade or destroy the product.

Methods and devices for controlling the activity and location of animals are also widely used. Often, these methods attempt to eliminate animals that have entered a space where they are not wanted, such as an attic or basement, using means such as poison or traps. These methods physically harm and often kill the animals, resulting in the need for removal and possibly disposal. Additionally, some methods of animal control may be considered inhumane. However, removal of such animals from spaces such as food storage warehouses, restaurants, and homes, is desirable since it minimizes disease and other unpleasant side effects of animal infestation in these areas.

Some animal control devices utilize scents to deter or repel an animal from an area. However, these products include only a few active ingredients and have a relatively short window of effectiveness. Additionally, such devices are only effective for one or two types of animals, meaning that multiple devices and/or compositions must be used in order to ensure all undesired species are not present in the space. Further, these types of deterrents may also have the appearance (and possibly scent) of being edible, thereby posing a risk of ingestion by other animals (e.g., house pets) or children. Often, the inert substrates used to hold the fragrance (e.g., paper, fibers, etc.) actually function as nesting material for the pest once the device is no longer effective thereby facilitating, rather than preventing, animal infestation.

Thus, a need exists for a long-lasting and robust fragrance-emitting article that does not pose a danger to animals or humans but is effective for deterring or repelling pests from any indoor or outdoor space or area. When used for controlling animal activity (e.g., as a repellant), it is desirable that the article be effective for more than one species and that it deter the target animals, rather than cause physical harm or death.

### SUMMARY

In one aspect, the present technology involves a fragrance-emitting article comprising: a solid substrate comprising a plurality of pores defined with the substrate; and a fragrant liquid drawn into at least a portion of the pores, wherein the fragrant liquid comprises (i) a mixture of three or more essential oils selected from the group consisting of garlic oil, peppermint oil, rosemary oil, clover oil, cinnamon oil, and cedarwood oil; or (ii) sandalwood oil.

In one aspect, the present technology involves a combination of essential oils used as an animal repellant, the combination comprising: (a) garlic oil in an amount of about 0.01 to about 50 weight percent; (b) peppermint oil in an amount of about 5 to about 65 weight percent; (c) rosemary oil in an amount of about 0.01 to about 50 weight percent; (d) clove oil in an amount of about 1 to about 60 weight percent; (e) cinnamon oil in an amount of about 0.5 to about 65 weight percent; and (f) cedarwood oil in an amount of about 0.5 to about 25 weight percent, wherein the weight of each oil is based on the total weight of the combination taken as 100 percent.

In one aspect, the present technology involves a method of making a fragrance-emitting article, the method comprising: (a) providing at least one solid porous substrate comprising a plurality of vesicles defined within the substrate; (b) providing a liquid comprising at least one fragrant compound; (c) contacting at least a portion of the solid porous substrate with the liquid; (d) depressurizing the liquid and/or the solid substrate to a pressure below atmospheric pressure; and (e) recovering a treated substrate at least partially saturated with the liquid and capable of emitting at least a portion of the fragrant compound to the surrounding environment.

### DETAILED DESCRIPTION

In this description, references to “one embodiment,” “an embodiment,” or “embodiments” mean that the feature or features referred to are included in at least one embodiment of the invention. Separate references to “one embodiment,” “an embodiment,” or “embodiments” in this description do not necessarily refer to the same embodiment and are not mutually exclusive unless so stated. Specifically, a feature, component, action, step, etc. described in one embodiment may also be included in other embodiments but is not necessarily included. Thus, particular implementations of the present invention can include a variety of combinations and/or integrations of the embodiments described herein.

According to embodiments of the present technology, there is provided a blend of essential oils particularly useful as an animal repellant. As used herein, the term “essential oil” refers to any natural oil (or synthetic version thereof) containing aromatic compounds derived from plants. Essential oils may be referred to as such when the oil carries the characteristic flavor and/or smell (essence) of the plant. Typically, essential oils are recovered from plants via distillation, but may be formed in any suitable manner such as, for example, chemical extraction or other technique. Each essential oil may include a variety of compounds, at least a portion of which may be aromatic and/or fragrant. As used herein, a “fragrant” compound refers to a compound having a volatility and aroma that can be detected by the olfactory system of a human or non-human animal. Essential oils as described herein may comprise one or more fragrant compounds.

Examples of suitable essential oils include, but are not limited to, agarwood, allspice, amyris, angelica root, anise, anise star, western red cedar, balm mint bush, balsam peru, basil, holy basil, lemon basil, bay leaf, bergamot, bergamot mint, birch, black pepper, black spruce, blue cypress, blue tansy, desert rosewood, white tea tree, camphor, hemp, caraway, cardamom, carrot seed, cassia, catnip, cedarwood (atlas, Himalayan, Texas, Virginian), celery seed, chamomile (German or Roman), cilantro, cinnamon, rock rose, citronella, sage, clementine, clove, coffee, cognac, copaiba, coriander, cumin, cypress, davana, dill, douglas fir, elemi, eucalyptus, helichrysum, fennel, finger root, balsam fir, Siberian fir, silver fir, fragonia, frankincense, galangal, galbanum, geranium, ginger, goldenrod, grapefruit, helichrysum, hemlock, ho leaf, ho wood, hyssop, jasmin, juniper, kumquat, kunzea, lavandin, lavender, ledum, lemon, lemongrass, lime, litsea, magnolia, mandarin, manuka, marjoram, tea tree, moldavian dragonhead, myrrh, myrtle, orange blossom, nootka tree, nutmeg, opoponax, oregano, palmarosa, patchouli, peppermint, petitgrain, pine, pink pepper, plai, Ravensara, rhododendron, rose, rosemary, rose wood, sage, sandalwood, saro, savory summer, savory winter, spearmint, spikenard, black spruce, tangerine, tarragon, thyme, turmeric, valerian, vanilla, verbena, vetiver, vitex, wintergreen, yarrow, ylang ylang, or yuzu. Combinations of one, two or more, three or more, four or more, five or more, or six of the above essential oils may be used according to embodiments of the present technology.

In some cases, essential oils suitable for use in blends according to embodiments of the present technology include garlic oil, peppermint oil, rosemary oil, clover oil, cinnamon oil, cedarwood oil, and combinations thereof. In some embodiments, the essential oil blend can comprise garlic oil in an amount of at least about 0.01, at least about 0.1, at least about 0.5, at least about 1, at least about 2, at least about 5, at least about 10, at least about 15, or at least about 20 weight percent and/or not more than about 50, not more than about 45, not more than about 40, not more than about 35, not more than about 30, not more than about 25, not more than about 20, not more than about 15, not more than about 12, not more than about 10, not more than about 8, not more than about 5, or not more than about 2.5 weight percent, based on the total weight of essential oils in the essential oil blend. Garlic oil may be present in an amount in the range of from about 1 to about 50, about 5 to about 50, about 10 to about 45, or about 20 to about 35 weight percent, or from about 0.1 to about 12, about 0.5 to about 10, or about 1 to about 5 weight percent, or from about 0.01 to about 10, about 0.1 to about 8, or about 0.5 to about 2.5 weight percent, based on the total weight of essential oils in the blend.

In some embodiments, the essential oil blend can include peppermint oil in an amount of at least about 1, at least about 2, at least about 5, at least about 10, at least about 15, at least about 20, at least about 25, or at least about 30 weight percent and/or not more than about 65, not more than about 60, not more than about 55, not more than about 50, not more than about 45, not more than about 40, not more than about 35, or not more than about 30 weight percent, based on the total weight of essential oils in the essential oil blend. Peppermint oil may be present in an amount in the range of from about 5 to about 65, about 5 to about 50, about 10 to about 45, or about 20 to about 35, from about 10 to about 60, or about 30 to about 50, from about 5 to about 60, about 10 to about 50, or about 20 to about 35 weight percent, based on the total weight of essential oils in the blend.

In some embodiments, the essential oil blend can comprise rosemary oil in an amount of at least about 0.01, at least about 0.1, at least about 0.5, at least about 1, at least about 2, at least about 5, at least about 10, at least about 15, or at least about 20 weight percent and/or not more than about 50, not more than about 45, not more than about 40, not more than about 35, not more than about 30, not more than about 25, not more than about 20, not more than about 15, not more than about 12, not more than about 10, not more than about 8, not more than about 5, or not more than about 2.5 weight percent, based on the total weight of essential oils in the essential oil blend. Rosemary oil may be present in the essential oil blend in an amount in the range of from about 0.1 to 50, about 1 to about 50, about 5 to about 50, about 10 to about 45, or about 20 to about 35, from about 0.1 to about 12, about 0.5 to about 10, or about 1 to about 5, or from about 0.01 to about 10, about 0.10 to about 8, or about 0.5 to about 2.5 weight percent, based on the total weight of essential oils in the blend.

In some embodiments, the essential oil blend can comprise clove oil in an amount of at least about 0.5, at least about 1, at least about 2, at least about 5, at least about 10, at least about 15, or at least about 20 weight percent and/or not more than about 60, not more than about 55, not more than about 50, not more than about 45, not more than about 40, not more than about 35, not more than about 30, not more than about 25, not more than about 20, not more than about 15, not more than about 12, not more than about 10, or not more than about 5 weight percent, based on the total weight of essential oils in the blend. Clove oil can be present in the blend in an amount in the range of from about 1 to about 60, about 1 to about 30, about 2 to about 25, or about 5 to about 20, from about 1 to about 25, about 2 to about 20, or about 5 to about 12, from about 5 to about 60, about 10 to about 50, or about 20 to about 35 weight percent, based on the total weight of essential oils in the essential oil blend.

In some embodiments, the essential oil blend may include cinnamon oil in an amount of at least about 0.1, at least about 0.5, at least about 1, at least about 2.5, at least about 5, at least about 10, at least about 15, or at least about 20 weight percent and/or not more than about 65, not more than about 60, not more than about 55, not more than about 50, not more than about 45, not more than about 40, not more than about 35, not more than about 30, not more than about 25, not more than about 20, not more than about 15, not more than about 10, or not more than about 5 weight percent, based on the total weight of essential oils in the essential oil blend. Cinnamon oil may be present in the essential oil blend in an amount in the range of from about 0.5 to 60, about 0.5 to about 20, about 1 to about 15, or about 2.5 to about 20, from about 5 to about 65, about 10 to about 60, or about 30 to about 50, from about 5 to about 60, about 10 to about 50, or about 20 to about 35 weight percent, based on the total weight of essential oils in the blend.

In some embodiments, the essential oil blend may comprise cedarwood oil in an amount of at least about 0.5, at least about 1, at least about 1.5, at least about 2, at least about 2.5, at least about 5, at least about 5.5, or at least about 7 weight percent and/or not more than about 30, not more than about 25, not more than about 20, not more than about 17.5, not more than about 15, not more than about 12, not more than about 10, not more than about 8, not more than about 7.5, or not more than about 5 weight percent, based on the total weight of essential oils in the essential oil blend. Cedarwood oil can be present in the essential oil blend in an amount in the range of from about 0.5 to 25, about 0.5 to about 20, about 1 to about 15, or about 2.5 to about 10, from about 1 to about 25, about 2 to about 20, or about 5 to about 12 weight percent, based on the total weight of essential oils in the essential oil blend.

In some embodiments, the essential oil composition may comprise sandalwood in an amount of at least about 90, at least about 95, at least about 97, or at least about 99 weight percent, based on the total weight of essential oils. In some cases, the only essential oil utilized may be sandalwood. In some embodiments, the essential oil composition can further include one or more fragrances that are the same or different than the scent of the essential oil.

The blend of essential oils may include at least two, at least three, at least four, at least five, or all of the above-listed essential oils combined with one another in varying amounts. The blend may include three or more, or four or more, or five, or all essential oils chosen from, or selected from the group consisting of, garlic oil, peppermint oil, rosemary oil, clover oil, cinnamon oil, cedarwood oil. In some cases, at least two, at least three, or four of the essential oils (including, for example, two, three, or four of garlic oil, peppermint oil, rosemary oil, clover oil, cinnamon oil, and cedarwood oil) in the blend can make up at least 50, at least 60, at least 70, at least 75, at least 80, at least 85, or at least 90 percent of the total blend, while the remaining components make up the balance. According to some embodiments, the essential oil blend of the present technology can include each of garlic oil, peppermint oil, rosemary oil, clove oil, cinnamon oil, and cedarwood oil, with the blend including less than about 0.5, less than about 0.1, less than about 0.05, less than about 0.01, less than about 0.005, or less than about 0.001 weight percent of any other oil or essential oil other than garlic oil, peppermint oil, rosemary oil, clove oil, cinnamon oil, and cedarwood oil. Additionally, or in the alternatively, the blend of essential oils can consist essentially of or consist of garlic oil, peppermint oil, rosemary oil, clover oil, cinnamon oil, and cedarwood oil.

Table 1, below, provides broad, intermediate, and narrow ranges for several exemplary essential oil blends formulated according to embodiments of the present invention.

TABLE 1

| | Formulation #1 (wt % of blend) | | |
|---|---|---|---|
| Essential Oil | Broad | Intermediate | Narrow |
| Garlic Oil | 5 to 50 | 10 to 45 | 20 to 35 |
| Peppermint Oil | 5 to 50 | 10 to 45 | 20 to 35 |
| Rosemary Oil | 5 to 50 | 10 to 45 | 20 to 35 |
| Clove Oil | 1 to 30 | 2 to 25 | 5 to 20 |
| Cinnamon Oil | 0.5 to 20 | 1 to 15 | 2.5 to 10 |
| Cedarwood Oil | 0.5 to 20 | 1 to 15 | 2.5 to 10 |
| | Formulation #2 (wt % of blend) | | |
| Essential Oil | Broad | Intermediate | Narrow |
| Garlic Oil | 0.1 to 12 | 0.5 to 10 | 1 to 5 |
| Peppermint Oil | 5 to 65 | 10 to 60 | 30 to 50 |
| Rosemary Oil | 0.1 to 12 | 0.5 to 10 | 1 to 5 |
| Clove Oil | 1 to 25 | 2 to 20 | 5 to 12 |
| Cinnamon Oil | 5 to 65 | 10 to 60 | 30 to 50 |
| Cedarwood Oil | 1 to 25 | 2 to 20 | 5 to 12 |
| | Formulation #3 (wt % of blend) | | |
| Essential Oil | Broad | Intermediate | Narrow |
| Garlic Oil | 0.01 to 10 | 0.1 to 8 | 0.5 to 2.5 |
| Peppermint Oil | 5 to 60 | 10 to 50 | 20 to 35 |
| Rosemary Oil | 0.01 to 10 | 0.1 to 8 | 0.5 to 2.5 |
| Clove Oil | 5 to 60 | 10 to 50 | 20 to 35 |
| Cinnamon Oil | 5 to 60 | 10 to 50 | 20 to 35 |
| Cedarwood Oil | 1 to 25 | 2 to 20 | 5 to 12 |

According to embodiments of the present invention, the essential oil blend may be used as an animal repellant capable of discouraging one or more species of animal from entering a space or area, if present, encouraging one or more species of animal to leave a space or area. Although not wishing to be bound by theory, it is believed that the combination of multiple (e.g., three or more) essential oils in the blend stimulates an olfactory over-stimulation in the target animal, prompting it to vacate or avoid the designated area or space. Although described herein with regard to natural essential oils, it should be understood that synthetic compounds having the same chemical profiles (and, particularly, the same or similar aromas) can be used with similar results.

The essential oil blend can be produced in any suitable manner such that the oils are homogeneously or generally homogenously blended. In some embodiments, the blend can be de-aerated by, for example, subjecting it to a vacuum to remove any residual dissolved gases and/or liquids such as water. Accordingly, the essential oil blend may comprise less than about 5, less than about 2, less than about 1, or less than about 0.5 percent of dissolved gases, based on the total weight of the blend.

According to embodiments of the present technology, there is provided a fragrance-emitting article formed using at least one fragrant compound such as, for example, a blend of essential oils as described herein. The fragrance-emitting article may comprise at least one solid substrate having a plurality of pores defined therein. As used herein, the term “pore” refers generally to any type of open space within a solid material or object, regardless of size or shape. The fragrance-emitting article further includes liquid drawn into at least a portion of the pores. The article is configured to emit at least one fragrant compound from the liquid within the pores into the surrounding environment. As mentioned previously, the fragrant compound may be volatile and detectable by the olfactory systems of a human or one or more non-human animal species.

Any suitable type of solid porous substrate may be used to form fragrance-emitting articles according to embodiments of the present invention. The substrate may be a naturally-occurring substrate or it may be synthetic. Examples of suitable substrates include, but are not limited to, porous rocks such a lava rocks including scoria, pumice, basalt, as well as other porous rocks such as sandstone, limestone, dolomite, porphyry, tuff, gneiss, diatomite, and gypsum. The rocks can be in any form, including powders, particles, chunks, or even rock wool. Alternatively, the substrate may comprise a porous material such as plastic, glass, carbon, ceramic, paper, plant fibers, sponge, cork, concrete, wood, and clay, as well as combinations thereof. The substrate itself may be inert and, in some embodiments, it may be reusable.

The average size of the pores within the substrate may be in the range of from about 0.5 microns to 2 mm, from about 1 micron to about 1 mm, from about 10 microns to about 0.5 mm, or from about 200 microns to about 350 microns. The total porosity, or open volume, within the substrate can be at least about 50, at least about 60, at least about 70, at least about 75, at least about 80, at least about 85, or at least about 90 percent, and/or not more than 99, not more than 97, not more than about 95, not more than about 90, not more than about 85, not more than about 80, or not more than about 75 percent. In some embodiments, the substrate can have a relatively high porosity, but a low permeability. As used herein, the term “porosity” refers to the total open volume of the substrate, while the term “permeability” refers to the amount of fluid capable of passing through the material per unit time. Lower permeability may permit the substrate to retain a larger volume of liquid and/or hold a volume of liquid longer within its pores.

The article may comprise a single substrate (such as a large rock or a piece of wood), or it may include multiple substrates (e.g., pieces of crushed rock, particles or beads of clay or plastic, or multiple fibers or other shaped elements). In some embodiments, the substrate has a three-dimensional shape and is not flat. In some embodiments, the substrate may be in the form of beads, particles, or fibers, while, in other embodiments, the substrate may have an irregular shape. The substrate may have a size (measured by longest dimension or, where multiple substrates are present, an average of longest dimensions of at least 10 substrates from a sample) of at least about 0.025, at least about 0.050, at least about 0.10, at least about 0.25, at least about 0.50, at least about 0.75, at least about 1, at least about 1.5, at least about 2, at least about 2.5, or at least about 3 inches and/or not more than about 24, not more than about 18, not more than about 15, not more than about 12, not more than about 10, not more than about 8, not more than about 6, not more than about 4, not more than about 3, or not more than about 2.5 inches. In some cases, the average substrate size can be less than about 1, less than about 0.75, less than about 0.5, less than about 0.25, less than about 0.10, or less than about 0.050 inches, measured as described above. In other cases, the average substrate size can be at least about 3, at least about 5, at least about 6, at least about 8, at least about 10, or at least about 12 inches, measured as described above.

Generally, the substrate may be non-toxic to plants, animals, and humans, and it may not be ingestible. One drawback associated with other scented articles is the ingestible nature (or perceived ingestible nature) of the article by humans (e.g., small children) or domestic animals (e.g., house pets). Articles of the present technology do not utilize edible or ingestible substrates and have a size, shape, and overall configuration that makes them undesirable to children and animals. Further, the substrate itself may not include any plant waste (e.g., corn cobs, plant fibers, etc.) and it may also not include any animal by-products (e.g., gastropod shells, mollusk shells, eggshells, etc.). In some embodiments, the substrate used for the fragrance-emitting article may include not more than about 0.5, not more than about 0.1, not more than about 0.05, not more than about 0.025, not more than about 0.01, not more than about 0.005, or not more than about 0.001 weight percent of one or more plant and/or animal waste or by-products. As a result, the materials used to form the substrate described herein are not capable of being utilized as nesting material for the animals intended to be deterred, when the fragrance-emitting articles described herein are used as an animal repellent. Additionally, in some embodiments, the substrate does not include more than about 0.5, not more than about 0.1, not more than about 0.05, not more than about 0.025, not more than about 0.01, not more than about 0.005, or not more than about 0.001 weight percent of any of limestone, chalk, calcium carbonate, paper, or paper-based or cloth-like fibers.

According to some embodiments of the present technology, the fragrant liquid drawn into the pores of the substrate can comprise at least one fragrant compound and may, for example, include an essential oil blend as described herein. Other examples include, but are not limited to, bodily fluids such as blood, chemicals, acids, and combinations thereof. When an essential oil blend as described herein is used, it may include at least three, at least four, at least five, or all essential oils listed above, with one or more or each being present in amounts within one or more of the ranges. Additionally, the liquid within the pores of the substrate in the fragrance-emitting article may also include water in an amount of at least about 1, at least about 2, at least about 5, or at least about 8 weight percent and/or not more than about 30, not more than about 25, not more than about 20, not more than about 15, not more than about 10, not more than about 5, or not more than about 2.5 weight percent, based on the total weight of the liquid. The liquid may be inert so that it does not chemically react with the substrate.

In some embodiments, the liquid applied to (and draw into the pores of) the substrate may include less than about 0.01, less than about 0.001, or less than about 0.0001 weight percent of one or more of one or more surfactants, one or more alcohols or glycols, one or more soaps, preservatives, thickeners, emulsifiers, other essential oils, or mineral oils. The liquid may consist essentially of, or consist of, the essential oil blend, with few or no other components.

Overall, the fragrance-emitting articles of the present invention may include at least about 10, at least about 15, at least about 20, or at least about 22 weight percent of the liquid and/or not more than about 50, not more than about 45, not more than about 40, not more than about 35, not more than about 30, not more than about 25, or not more than about 20 weight percent of the liquid, based on the total weight of the article. All or a portion of the components present in the liquid may be considered an active ingredient. The substrate may account for at least about 50, at least about 55, at least about 60, at least about 65, at least about 70, at least about 75, or at least about 80 percent of the article and/or not more than about 90, not more than about 85, not more than about 80, or not more than about 78 weight percent of the article.

In some embodiments, the fragrance-emitting article can include garlic oil in an amount in the range of from about 1 to 12, about 2 to about 8, about 2.5 to about 7.5, or from about 0.5 to about 10, about 1 to about 7.5, or about 2 to about 5 weight percent, based on the total weight of the article.

In some embodiments, the fragrance emitting article may comprise peppermint oil in an amount in the range of from about 1 to about 12, about 2 to about 8, or about 2.5 to about 7.5, from about 2 to about 25, about 5 to about 20, or about 7.5 to about 15 weight percent, or about 1 to about 20, about 2.5 to about 15, or about 5 to about 10 weight percent, based on the total weight of the article.

In some embodiments, the fragrance-emitting article may comprise rosemary oil in an amount in the range of from about 1 to about 12, about 2 to about 8, or about 2.5 to about 7.5, from about 0.5 to about 10, about 1 to about 7.5, or about 2 to about 5 weight percent, based on the total weight of the article.

In some embodiments, the fragrance-emitting article can comprise clove oil in an amount in the range of from about 0.5 to about 10, about 1 to about 7.5, or about 2 to about 5, from about 0.1 to 7.5, about 0.5 to about 5, or about 0.75 to about 2.5, from about 1 to about 20, about 2.5 to about 15, or about 5 to about 10 weight percent, based on the total weight of the article.

In some embodiments, the fragrance-emitting article can comprise cinnamon oil in an amount of from about 0.1 to about 7.5 weight percent, about 0.5 to about 5 weight percent, or about 0.75 to about 2.5 weight percent, or from about 2 to about 25 weight percent, about 5 to about 20 percent, or about 7.5 to about 15 weight percent, from about 1 to about 20 weight percent, about 2.5 to about 15 weight percent, or about 5 to about 10 weight percent, based on the total weight of the article.

In some embodiments, the fragrance-emitting article can comprise cedarwood oil in an amount of from about 0.1 to about 7.5 weight percent, about 0.5 to about 5 weight percent, or about 0.75 to about 2.5 weight percent, based on the total weight of the article.

Broad, intermediate, and narrow ranges for amounts of several essential oils present in exemplary articles according to embodiments of the present technology are provided in Table 2 below. The values provided in Table 2 are based on the article as a whole (substrate and all liquid) taken as being 100 percent.

TABLE 2

| | Formulation #1 (wt % of article) | | |
|---|---|---|---|
| Essential Oil | Broad | Intermediate | Narrow |
| Garlic Oil | 1 to 12 | 2 to 8 | 2.5 to 7.5 |
| Peppermint Oil | 1 to 12 | 2 to 8 | 2.5 to 7.5 |
| Rosemary Oil | 1 to 12 | 2 to 8 | 2.5 to 7.5 |
| Clove Oil | 0.5 to 10 | 1 to 7.5 | 2 to 5 |
| Cinnamon Oil | 0.1 to 7.5 | 0.5 to 5 | 0.75 to 2.5 |
| Cedarwood Oil | 0.1 to 7.5 | 0.5 to 5 | 0.75 to 2.5 |
| | Formulation #2 (wt % of article) | | |
| Essential Oil | Broad | Intermediate | Narrow |
| Garlic Oil | 1 to 12 | 2 to 8 | 2.5 to 7.5 |
| Peppermint Oil | 2 to 25 | 5 to 20 | 7.5 to 15 |
| Rosemary Oil | 1 to 12 | 2 to 8 | 2.5 to 7.5 |
| Clove Oil | 0.1 to 7.5 | 0.5 to 5 | 0.75 to 2.5 |
| Cinnamon Oil | 2 to 25 | 5 to 20 | 7.5 to 15 |
| Cedarwood Oil | 0.1 to 7.5 | 0.5 to 5 | 0.75 to 2.5 |
| | Formulation #3 (wt % of article) | | |
| Essential Oil | Broad | Intermediate | Narrow |
| Garlic Oil | 0.5 to 10 | 1 to 7.5 | 2 to 5 |
| Peppermint Oil | 1 to 20 | 2.5 to 15 | 5 to 10 |
| Rosemary Oil | 0.5 to 10 | 1 to 7.5 | 2 to 5 |
| Clove Oil | 1 to 20 | 2.5 to 15 | 5 to 10 |
| Cinnamon Oil | 1 to 20 | 2.5 to 15 | 5 to 10 |
| Cedarwood Oil | 0.1 to 7.5 | 0.5 to 5 | 0.75 to 2.5 |

According to some embodiments of the present technology, the article may further include at least one solid, or semi-solid component applied to the exterior surface of the porous substrate. Such component may be in addition to, or alternatively to, the liquid drawn into the pores of the substrate. The solid or semi-solid component may be in the form of a powder, a gel, a slurry of particles, or other form suitable for application to the substrate. When applied, the solid may form an outer shell or layer on the substrate having an average thickness of, for example, not more than about 2 mm, not more than about 1 mm, not more than about 0.5 mm, or not more than about 0.25 mm, measured at five locations along the surface of the substrate. In other embodiments, a liquid including, for example, a water-based liquid, an oil-based liquid, or an emulsion or homogeneous blend of oil and water may additionally, or alternatively, be applied to the outer surface of the solid substrate after the liquid has been drawn into the pores. This may help slow down diffusion of the fragrant composition into the environment and/or prevent decay or drying of the inner fragrance, particularly when the inner liquid is water-based or includes significant amounts of water.

In some embodiments, the liquid applied to the outer surface of the at least partially saturated solid substrate may also comprise an active ingredient. One example of this is a porous substrate including a liquid with garlic oil drawn into the pores and a liquid comprising peppermint oil applied to the outer surface. When present, the material applied to the outer surface of the substrate can be solid, semi-solid, or liquid, or combinations thereof. In some embodiments, the material applied to the outer surface of the substrate can be similar to or the same as the material drawn into the pores of the substrate. For example, the outer coating may be applied by combining a powder binder with the scented oil (e.g., essential oil) and applying the mixture to the outer surface of the substrate. Optionally, the liquid used to apply the material can include additional components, such as alcohol, water, or combinations of these components.

According to embodiments of the present technology, methods for making fragrance-emitting articles as described herein are also provided. In some cases, the methods may include providing at least one solid porous material (e.g., substrate) having a plurality of pores as described previously and providing at least one liquid comprising one or more fragrant compounds as also described previously. The liquid and solid may be contacted such as, for example, by spraying, brushing, or otherwise coating the liquid onto the surface of the solid (or solids) or by partially or completely submerging the solid (or solids) in the liquid. In some cases, the submerged (or partially submerged) solid may then be allowed to soak up the liquid, optionally with agitation, for a time period of at least about 1, at least about 5, at least about 10, at least about 15, or at least about 20 hours and/or not more than about 40, not more than about 25, or not more than about 20 hours. In some cases, sonication or other type of vibration may be used to help facilitate placement of the liquid into the pores of the solid.

Alternatively, or in addition, the liquid and/or solid can be pressurized so that, for example, one or both are exposed to pressures above atmospheric. In some embodiments, the solid may be submerged (partially or completely) in the liquid, and the resulting slurry can be subjected to pressures at least about 2, at least about 5, at least about 8, or at least about 10 psi above atmospheric pressure and/or not more than about 50, not more than about 40, not more than about 30, not more than about 20, or not more than about 15 psi above atmospheric pressure. Any suitable method of achieving such a pressurization may be used.

Alternatively, or in addition, the liquid and/or solid can be de-pressurized so that, for example, one or both are exposed to pressures below atmospheric pressure. For example, in some embodiments, the liquid may be subjected to pressures that are at least about 2, at least about 5, at least about 8, or at least about 10 psi below atmospheric pressure, whereafter the depressurized liquid may then contact the solid substrate. In some cases, the solid may be submerged or partially submerged and the resulting slurry depressurized to a pressure within one or more ranges above. Such depressurization may help draw liquid into the pores of the substrate by, for example, removing air and other liquids from the pores of the substrate and permitting the surrounding liquid to flow into the solid material. Any suitable method of applying a vacuum to the solid and liquid slurry, or the liquid itself, may be used. In some cases, the vacuum can be alternately applied and released until no further air is being removed (e.g., no bubbles are evident in the liquid). In some embodiments, exposing the liquid and/or solid to vacuum may help prevent undesired breakdown of the essential oil(s) into aldehydes, ketones, and free fatty acids.

In some embodiments of the present technology, the solid materials used as the substrate for the fragrance-emitting article may be reduced in size prior to being contacted with the fragrant liquid. For example, solid particles or materials may first be crushed, ground, broken, or otherwise made smaller to provide solids having an average size within the ranges describe above. In other cases, larger particles may be subjected to the contacting (and, pressurizing and/or depressurizing steps, where applicable) and the resulting treated particles recovered therefrom may then be crushed, ground, or otherwise broken into smaller particles having a size within the ranges mentioned above.

The treated particles, which can be at least partially saturated with the liquid and capable of emitting at least a portion of the fragrant compound into the surrounding environment, may exhibit an overall weight increase of at least about 5, at least about 7.5, at least about 10, at least about 12.5, or at least about 15 weight percent and/or not more than about 55, not more than about 50, not more than about 45, not more than about 40, not more than about 35, not more than about 30, not more than about 25, or not more than about 20 weight percent, measured according to the following formula: (Weight of Substrate After Treatment−Weight of Substrate Before Treatment)/Weight of Substrate Before Treatment, expressed as a percentage.

The at least partially saturated fragrance-emitting articles may then be recovered and further processed for end use. Examples of additional processing steps include, but are not limited to, drying and packaging the fragrance-emitting articles. In some cases, the packaging can include packaging a single article, while in other cases, it includes packaging a plurality of articles. In some cases, the fragrance-emitting solids may also be further size-reduced, if needed.

According to some embodiments of the present technology, the saturated article may be considered a slow-release article or composition. This can be measured by, for example, fully saturating a substrate with a desired liquid, then permitting the substrate to remain in an indoor space under ambient temperature and pressure in the absence of any excessive UV light exposure and no wind. After one hour under these conditions, if the article exhibits a weight loss of 10 percent or less, calculated as described above, it may be considered a slow-release article. Articles of the present technology typically have an active life of at least about 10, at least about 20, at least about 30, at least about 45, at least about 60, at least about 75 days, at least about 80 days, or at least about 90 days. Active life is calculated by the time it takes for the weight of the treated substrate to fall within 0.5% of its original weight prior to treatment with the fragrant liquid.

According to embodiments of the present technology, the fragrance-emitting articles described herein may be used as an animal repellant in an indoor or outdoor space that is enclosed or open. The method for repelling or deterring animals from a space or area includes placing or applying one or more fragrance-emitting articles as described herein into the space and exposing all or a portion of the fragrance-emitting article to air or air flow within the space. Accordingly, volatile, fragrant compounds from the liquid within the pores of the article will at least partially vaporize and may be carried throughout the space.

In some embodiments, the fragrance-emitting article may be a repellant for a single species of animal, while, in other embodiments, the articles may be a repellant for multiple species of animals. As used herein, the term “animal” refers to any non-human multi-cellular organism, including, for example, reptiles, amphibians, insects, and mammals. Specific examples of species for which articles according to embodiments of the present technology may be useful as repellants include, but are not limited to, arachnids, chilopods, diplopods, arthropods, snakes, reptiles, birds, ruminants, rodents (e.g., mice and squirrels), as well as various other types of mammals, including domesticated mammals (e.g., dogs, cats, etc.). The animals repelled or deterred by the fragrance-emitting articles may be wild animals, domesticated animals, or livestock. In some embodiments, the animals deterred by compositions according to embodiments of the present invention include mice, rats, squirrels, ground hogs, moles, raccoons, possums, and combinations thereof. Other examples include, but are not limited to, deer, elk, moose, coyotes, wolves, and bobcats.

In some embodiments, the fragrance-emitting articles described herein may be used as an attractant and can be at least partially saturated with a fragrance designed to attract, rather than repel, one or more species of animals. Such articles may be used to draw animals to one area, thereby indirectly preventing them from entering another area.

In addition to utilizing fragrance-emitting articles including a liquid-saturated substrate as described herein, embodiments of the present technology also include other methods of applying a fragrance to an area such as, for example, spraying, misting, sprinkling, diffusing, or any combination thereof. Such methods may be used to, for example, apply a fragrant composition including a blend of essential oils as described herein. In some cases, however, use of a porous substrate may be preferable, since articles including such a component are more resistant to environmental conditions such as rain, snow, heat, wind, as well as UV light. Fragrant composition for animal control (e.g., repelling or attracting) positioned in an area or space in a porous substrate may have an overall effective life of at least about 20, at least about 25, at least about 30, or at least about 35 days and/or not more than about 100, not more than about 95, or not more than about 90 days. In contrast, fragrant compositions applied to an area by other methods may have a shorter effective life.

Compositions and articles of the present technology may be used in indoors or outdoors and in open areas or enclosed spaces. Examples of suitable locations where these components may be used for repelling and/or attracting animals include, but are not limited to, residential buildings such as homes, sheds, and garages, commercial buildings such as office spaces, warehouses, factories, and storage areas, including food storage areas, vehicles such as trucks, trains, cars, boats, RV/campers, farm equipment, industrial or commercial machines such as excavators, fork lifts, etc. Additionally, compositions and articles of the present technology can be used in inhabited areas, including food service and storage areas, grocery stores, restaurants, etc., as well as areas such as nurseries and hospitals.

The coverage area of articles of the present technology can vary depending on the amount of material used. Examples of suitable coverage areas include, but are not limited to, at least 25, at least 50, at least 100, at least 200, at least 300, or at least 400 square feet for 2.5 ounces of the fragrance-emitting article. In some cases, an open (or partially open) package of fragrance-emitting articles as described herein may be placed in a space to be treated. For example, a 2.5 ounce package of fragrance-emitting article may be at least partially opened to varying degrees to treat different coverage areas. Table 3, below summarizes some particular open percentages for the package as well as a corresponding treatment area. It should also be noted that extended active life may be achieved by opening the packaging less (smaller percentage open).

TABLE 3

| | Coverage Area, |
|---|---|
| Package Opening | Sq. Ft. |
| 6% | 25 |
| 12% | 50 |
| 25% | 100 |
| 50% | 200 |
| 75% | 300 |
| 100% | 400 |

Specific end uses for fragrance-emitting articles described herein include, for example, use of the article in an enclosed bird feeder to keep insects and squirrels away from the birds' food, but to prevent chemical deterrents (e.g., chili or habanero powder) from getting into the birds' eyes. As a further example, fragrance-emitting articles as described herein may be placed in a warehouse or other storage area and may drive out rodents, such as mice and rats, while still providing an environment safe for humans to work.

Benefits of the present technology include effective animal repellant that does not harm or kill the target animals. Not only is this more humane, but it also avoids unpleasant consequences of fatal pest control methods, such as foul odors and need to locate and dispose of deceased animals. Further, the substrates of the present technology are reusable, if desired, and, at minimum cannot be used as nesting material for the animals the articles are designed to repel. None of the components are toxic and most are naturally-occurring but are not ingestible by humans or other animals.

### Example

Pumice stone was crushed to an average size between 0.375 and 0.75 inches and it was saturated with a liquid including garlic oil (6.250 wt. %), cinnamon oil (1.250 wt. %), rosemary oil (6.250 wt. %), peppermint oil (6.250 wt. %), clover oil (3.750 wt. %), and cedarwood oil (1.250 wt. %). The crushed pumice was submerged in a liquid including the essential oil blend, and the slurry was subjected to a vacuum pressure of 29 inches of Hg vacuum for four hours. The resulting product was recovered and dried before being packaged in 2.5 ounce containers.

Five 2.5-ounce containers were placed in an 1800-square foot warehouse that contained pallets of straw, hay, bird seed, and other bulk items normally found in a retail hardware store. There was evidence of mouse infestation. The containers were placed along three walls opposite of the end wall where a 36-inch walk-in door and an eight-foot garage door with a broken floor seal were apparently providing access to mice to enter the warehouse. A camera was set to record the doors and was set to record for approximately 60 seconds when activated and shut off approximately 40 seconds thereafter to save battery life. Over the course of seven days, 139 mice were recorded leaving the area, during that time some mice would re-enter the warehouse but all were seen to immediately exit during the recording. No dead mice were reported by the warehouse owner during or after the test period. Leaving the product in place for 10 months, no mice or evidence of infestation were observed, even though the walk-in and garage doors were intermittently open during and after the test in order to move materials in and out of the warehouse.

### Definitions

As used herein, the terms “a,” “an,” and “the” mean one or more.

As used herein, the term “animal” refers to any non-human multi-cellular organism.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “essential oil” refers to any natural oil (or synthetic version thereof) containing aromatic compounds derived from plants.

As used herein, a “fragrant” compound refers to a compound having a volatility and aroma that can be detected by the olfactory system of a human or non-human animal.

As used herein, the terms “having,” “has,” and “have” have the same open-ended meaning as “comprising,” “comprises,” and “comprise” provided above.

As used herein, the terms “including,” “include,” and “included” have the same open-ended meaning as “comprising,” “comprises,” and “comprise” provided above.

As used herein, the phrase “at least a portion” includes at least a portion and up to and including the entire amount or time period.

As used herein, the term “permeability” refers to the amount of fluid capable of passing through the material per unit time.

As used herein, the term “porosity” refers to the total open volume of the substrate.

As used herein, the term “pore” refers generally to any type of open space within a solid material or object, regardless of size or shape.

### CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

What is claimed is:

1. A fragrance-emitting article comprising:
a solid substrate comprising a plurality of pores defined within the substrate; and
a fragrant liquid drawn into at least a portion of said pores, wherein the fragrant liquid comprises (i) a mixture of three or more essential oils selected from the group consisting of garlic oil, peppermint oil, rosemary oil, clover oil, cinnamon oil, and cedarwood oil; or (ii) sandalwood oil.

2. The article of claim 1, wherein the fragrant liquid comprises a mixture of each of garlic oil, peppermint oil, rosemary oil, clover oil, cinnamon oil, and cedarwood oil.

3. The article of claim 2, wherein the article includes garlic oil in an amount in the range of from about 2.5 to about 7.5 weight percent, peppermint oil in an amount in the range of from about 2.5 to about 7.5 weight percent, rosemary oil in an amount in the range of from about 2.5 to about 7.5 weight percent, clove oil in an amount in the range of from about 2 to about 5 weight percent, cinnamon oil in an amount in the range of from about 0.75 to about 2.5 weight percent, and cedarwood oil in an amount in the range of from about 0.75 to about 2.5 weight percent, based on the total weight of the article.

4. The article of claim 2, wherein the article includes garlic oil in an amount in the range of from about 2.5 to about 7.5 weight percent, peppermint oil in an amount in the range of from about 7.5 to about 15 weight percent, rosemary oil in an amount in the range of from about 2.5 to about 7.5 weight percent, clove oil in an amount in the range of from about 0.75 to about 2.5 weight percent, cinnamon oil in an amount in the range of from about 7 to about 15 weight percent, and cedarwood oil in an amount in the range of from about 0.75 to about 2.5 weight percent, based on the total weight of the article.

5. The article of claim 2, wherein the article includes garlic oil in an amount in the range of from about 2 to about 5 weight percent, peppermint oil in an amount in the range of from about 5 to about 10 weight percent, rosemary oil in an amount in the range of from about 2 to about 5 weight percent, clove oil in an amount in the range of from about 5 to about 10 weight percent, cinnamon oil in an amount in the range of from about 5 to about 10 weight percent, and cedarwood oil in an amount in the range of from about 0.75 to about 2.5 weight percent, based on the total weight of the article.

6. The article of claim 1, wherein the fragrant liquid comprises less than about 0.5 weight percent of each of surfactants, alcohols, polyols, soaps, preservatives, emulsifiers, mineral oil, castor oil, and essential oils other than garlic oil, peppermint oil, rosemary oil, cinnamon oil, clove oil, and cedarwood oil.

7. The article of claim 1, wherein the fragrant liquid comprises sandalwood oil.

8. The article of claim 1, wherein the substrate comprises porous lava rock.

9. The article of claim 1, wherein the article comprises another solid or liquid material coated onto at least a portion of an outer surface of the substrate, wherein the another solid or liquid is different than the fragrant liquid within the pores of the article.

9. An animal repellant composition comprising:
a blend of essential oils comprising—
(a) garlic oil in an amount of about 0.01 to about 50 weight percent;
(b) peppermint oil in an amount of about 5 to about 65 weight percent;
(c) rosemary oil in an amount of about 0.01 to about 50 weight percent;
(d) clove oil in an amount of about 1 to about 60 weight percent;
(e) cinnamon oil in an amount of about 0.5 to about 65 weight percent; and
(f) cedarwood oil in an amount of about 0.5 to about 25 weight percent,
wherein the weight percentages are based on the total weight of the blend of essential oils taken as 100 percent.

10. The composition of claim 9, wherein the composition includes garlic oil in an amount in the range of from about 10 to about 45 weight percent, peppermint oil in an amount in the range of from about 10 to about 45 weight percent, rosemary oil in an amount in the range of from about 10 to about 45 weight percent, clove oil in an amount in the range of from about 2 to about 25 weight percent, cinnamon oil in an amount in the range of from about 1 to about 15 weight percent, and cedarwood oil in an amount in the range of from about 1 to about 15 weight percent, based on the total weight of the essential oil blend.

11. The composition of claim 9, wherein the composition includes garlic oil in an amount in the range of from about 0.5 to about 10 weight percent, peppermint oil in an amount in the range of from about 10 to about 60 weight percent, rosemary oil in an amount in the range of from about 0.5 to about 10 weight percent, clove oil in an amount in the range of from about 2 to about 20 weight percent, cinnamon oil in an amount in the range of from about 10 to about 60 weight percent, and cedarwood oil in an amount in the range of from about 2 to about 20 weight percent, based on the total weight of the essential oil blend.

12. The composition of claim 9, wherein the composition includes garlic oil in an amount in the range of from about 0.5 to about 10 weight percent, peppermint oil in an amount in the range of from about 10 to about 60 weight percent, rosemary oil in an amount in the range of from about 0.5 to about 10 weight percent, clove oil in an amount in the range of from about 2 to about 20 weight percent, cinnamon oil in an amount in the range of from about 10 to about 60 weight percent, and cedarwood oil in an amount in the range of from about 2 to about 20 weight percent, based on the total weight of the essential oil blend.

13. The composition of claim 9, wherein the composition includes garlic oil in an amount in the range of from about 0.1 to about 8 weight percent, peppermint oil in an amount in the range of from about 10 to about 50 weight percent, rosemary oil in an amount in the range of from about 0.1 to about 8 weight percent, clove oil in an amount in the range of from about 10 to about 50 weight percent, cinnamon oil in an amount in the range of from about 10 to about 50 weight percent, and cedarwood oil in an amount in the range of from about 2 to about 20 weight percent, based on the total weight of the essential oil blend.

14. A method of making a fragrance-emitting composition, said method comprising:
(a) providing at least one solid substrate comprising a plurality of pores defined therein;
(b) providing a liquid comprising at least one fragrant compound;
(c) contacting at least a portion of the solid substrate with the liquid;
(d) depressurizing the liquid and/or the solid substrate to a pressure below atmospheric pressure; and
(e) recovering a treated substrate at least partially saturated with the liquid and capable of emitting at least a portion of the fragrant compound to a surrounding environment.

15. The method of claim 14, wherein the depressurizing of step (d) comprises depressurizing the liquid to a pressure below atmospheric pressure to provide a depressurized liquid and then adding the solid substrate to the depressurized liquid.

16. The method of claim 14, wherein the depressurizing of step (d) comprises adding the solid substrate to the liquid to provide an at least partially submerged substrate and depressurizing the at least partially submerged substrate.

17. The method of claim 14, wherein the providing of step (a) includes reducing the size of larger solid particles to provide the solid substrate, and wherein the solid substrate comprises a plurality of smaller sized solid particles.

18. The method of claim 14, wherein the recovering of step (e) includes packaging a plurality of treated substrates to provide a packaged article.

19. The method of claim 14, wherein the solid substrate comprises a plurality of solid particles, wherein the solid particles comprise pumice, and wherein the liquid comprises a blend of at least four essential oils.

20. The method of claim 14, wherein the treated substrate recovered in step (e) exhibits a weight increase of at least 5 percent as compared to the weight of the solid substrate provided in step (a).
